(19) United States
(12) Patent Application Publication
(10) Pub. No.: US 2017/0281004 A1
(43) Pub. Date: Oct. 5, 2017

(54) PERFORMING AN EXAMINATION

(71) Applicants: Claudia Igney, Erlangen (DE); Markus Petsch, Erlangen (DE); Jörg Christopher Stapf, Uttenreuth (DE)

(72) Inventors: Claudia Igney, Erlangen (DE); Markus Petsch, Erlangen (DE); Jörg Christopher Stapf, Uttenreuth (DE)

(21) Appl. No.: 15/477,088

(22) Filed: Apr. 1, 2017

(30) Foreign Application Priority Data
Apr. 1, 2016 (DE) 102016205459.7

(51) Int. Cl. A61B 5/00; A61B 5/055; G01R 33/341; G01R 33/48; G01R 33/30

(52) U.S. Cl. CPC A61B 5/0035; G01R 33/4808; G01R 33/307; G01R 33/341; A61B 5/055; A61B 5/704; A61B 5/004

(57) ABSTRACT

A method for performing an examination includes at least one magnetic resonance-based and at least one further examination step. An examination object is positioned during the entire examination on an upper side of a transfer plate that is positioned on an object table during the magnetic resonance-based examination. At least one coil holder that includes at least one local coil or by which at least one coil device is supported is used during the magnetic resonance-based examination step. The coil holder is arranged on the examination object during the magnetic resonance-based examination step exclusively. The coil holder is supported during the magnetic resonance-based examination steps by at least one mounting, into which a supporting section of the coil holder engages and/or which engages into the supporting section of the coil holder.

This application claims the benefit of DE 10 2016 205 459.7, filed on Apr. 1, 2016, which is hereby incorporated by reference in its entirety.

### BACKGROUND

The present embodiments relate to a method for performing an examination.

In medical imaging (e.g., in examinations, in angiography), a plurality of different measurement methods that are carried out with different devices are sometimes used. For example, MRI, MRI-PET, AX, CT-PET and/or CT devices are used in various examination steps. In magnetic resonance-based examination steps, a plurality of local coils are typically intended to be arranged on the examination object for this purpose. However, the corresponding local coils may first interfere with other examination steps, X-ray-based examinations, for example, and second, may impede the access to the examination object. This is particularly problematic in imaging in the area of the head.

For imaging the head in magnetic resonance-based examination steps, head coils have been used, as also used in MR only or MR-PET scanning. For example, a flexible housing that contains a plurality of coils (e.g., a Flex coil) may be wound around a head of a patient and affixed thereto. The problem here is that to apply such a head coil to the head of the patient, the head is to be moved. This may impair the comparability of the measurement data from the individual examination steps and may take up considerable time (e.g., when a patient is sedated).

### SUMMARY AND DESCRIPTION

The scope of the present invention is defined solely by the appended claims and is not affected to any degree by the statements within this summary.

The present embodiments may obviate one or more of the drawbacks or limitations in the related art. For example, a method for performing a multimodal examination, in which the arrangement of local coils on the examination object (e.g., head coils) is possible with little effort, where movement of the examination object is avoided, is provided.

The coil holder is supported during the magnetic resonance-based examination step by at least one mounting, into which a supporting section of the coil holder engages and/or which engages into the supporting section of the coil holder. The mounting is configured as part of the object table or of the transfer plate or of a supporting device that is arranged between the object table and the transfer plate.

According to one or more of the present embodiments, mountings may be provided for a coil holder that either directly or indirectly supports the local coils. The mountings are arranged on the object table, on the transfer plate, or a supporting device between them. As a result thereof, first, the coil holder and hence the local coils are supported in a defined position. Second, these mountings make it possible to arrange the coil holder and hence the local coils on the examination object in various ways, depending on their physical design, without having to change the patient's positioning on the transfer plate. As will be explained more precisely hereinafter, the coil holder may, for example, be guided from above in the direction of the examination object, enclose it laterally and engage with supporting sections into the mountings, which are arranged laterally to the examination object, on the transfer plate, the object table and/or the supporting device. It is also possible, even before positioning the coil holder on the transfer plate, to secure it on the patient couch on mountings on the object table or on a supporting device that is positioned on the object table and to subsequently position the transfer plate on the transfer table. In this case, coil holders can also be fitted into an area underneath the examination object in a particularly simple manner. To surround the examination object, the previously attached coil holders can, for example, be bent round the examination object, a patient for example, and affixed or additional coil holders or such like can be put on.

The method according to one or more of the present embodiments allows a simple attachment of the local coils to the examination object prior to the magnetic resonance-based examination step and/or an easy removal of the local coils after the magnetic resonance-based examination step. The various examination steps can preferably be carried out on object tables that differ from one another. The arrangement of the coil holder on the examination object can preferably ensue such that at least one of the local coils is located underneath the examination object. The mounting can be a recess, in which the coil holder is supported using a force-fit. A force-fit can be possible in particular as a result of the coil holder being elastic and being pressed against a wall of the recess by an elastic restoring force. It is also possible that the recess may have an undercut area into which the supporting section can engage, with a hook for example, in order to achieve a positive-fit connection. Conversely, it is also possible for the support to be designed as a protrusion that engages into a recess in the coil holder.

Preferably, at least two mountings are used which jointly support a coil holder or each support a coil holder. One of the supports can be accessible from the left-hand side and one from the right hand side of the examination object, such that the one coil holder or the two coil holders together can be guided over the examination object, in an arc shape, for example.

The coil holder and/or the coil device can be rigid or elastically flexible. The coil holder and/or the coil device can be dimensionally stable, in particular dimensionally stable in an arc shape, and preferably mechanically stable and/or self-supporting.

The coil holder or the coil device can be designed to be multi-channel, for example four-channel, six-channel, or eight-channel, that is, include a plurality of measurement coils that can be used individually or in various combinations by the magnetic resonance device.

The transfer plate can have one or a plurality of parts. It can consist of a base and or a fixed or removable patient mattress arranged thereon. The base can have one or a plurality of parts and in particular comprise a fixed or removable head area or a head rest to support a patient's head.

In the method according to one or more of the present embodiments, a mounting can be used that is configured on a side surface that differs from the upper surface of the transfer plate. Preferably, mountings can be used on opposite side surfaces of the transfer plate. A corresponding arrangement of the mounting makes it possible to arrange the coil holder on the transfer plate and hence on the examination object even before arranging the transfer plate on the object table. This can increase the efficiency in running the scan. Since the mounting is arranged on the side surface, however, the entire upper surface of the transfer plate is useable for the examination object without covering the mounting.

The coil holder can be arranged on the examination object prior to the magnetic resonance-based examination step, by its being guided vertically or at an angle to the surface of the transfer plate in the direction of the examination object, its supporting section being inserted into the mounting or the mounting being inserted into the supporting section. For example, an arc-shaped coil holder can be placed on the transfer plate or on the object table and supported by the mounting or the mountings once it has thus been put in place.

The mounting and the supporting section can be connected by a releasable locking connection. Corresponding locking connections are known in the prior art, for example, the SNAPLOC®-System. This ensures a reliable fastening of the coil holder onto the mounting, at the same time ensuring a simple release. Additionally or alternatively, it is possible for the mounting to be designed in the shape of a groove, into which the supporting section engages.

Prior to the magnetic resonance-based examination step, the coil holder in the mounting or the mounting with the coil holder supported thereon can be moved parallel to the transfer plate with respect to the examination object. For example, an arc-shaped coil holder can be fastened onto the object table, yet first arranged in a position that allows a problem-free positioning of the transfer plate on the object table. After positioning the transfer plate on the object table, the arc-shaped coil holder can then be moved, such that it includes a section of the examination object, the head, for example. The sliding of the coil holder can be facilitated using moveable mountings. Sliding the coil holder inside the mounting can facilitate such a sliding movement in a particularly simple manner, for example, if the mounting is configured as a groove, into which the supporting section engages, or if the supporting section has a groove, into which the mounting engages. In the method according to one or more of the present embodiments, a sliding movement with respect to the examination object can also correspond to a sliding movement with respect to the transfer plate. If the transfer plate is positioned on the object table, a sliding movement with respect to the object table therefore also ensues.

Prior to the magnetic resonance-based examination step, at least one free section of the coil holder and/or of the coil device can be bent and fixed such that the coil holder and/or the coil device alone and/or together with the object table and/or the transfer plate encloses or enclose the examination object during the magnetic resonance-based examination step. What is meant by enclosing is in particular encompassing, in which a section of the examination object, is surrounded for example in a circular manner, by the enclosing elements, yet protrudes on one side into a longitudinal direction or protrudes out of the surrounded area on both sides. Enclosing, can be achieved for example, by a coil holder, which is affixed in each case to a lateral supporting section, being provided for each side of the examination object, wherein the two coil holders are brought together in an arc over the examination object and fixed to one another or to a third object above the examination object. It is also possible for a coil holder to be used that is affixed on one side to a mounting, is guided in particular in an arc over the examination object and affixed on the other side of the examination object, preferably, on a further mounting. If the mountings are configured such that the supported coil holder also extends underneath the examination object, with this procedure a similar scan geometry can be achieved as when a flexible coil is wound directly round the examination object, in which case no repositioning of the examination object is necessary in the method according to one or more of the present embodiments.

The coil holder and/or the coil device can be arranged on the examination object such that the coil holder and/or the coil device alone and/or together with the object table and/or the transfer plate includes or include the examination object during the magnetic resonance-based examination step, wherein, on the side of the transfer plate that is turned away from the examination object, a predetermined free volume is maintained between the examination object and the coil holder and/or the coil device. A corresponding free volume can, for example, be a space sufficient to provide tubes to intubate the examination object. A free volume can be provided, for example, by using in particular an elastically dimensionally stable, arc-shaped coil holder or an in particular elastically dimensionally stable arc-shaped coil device, or by the coil holder and/or the coil device being supported by a spacer.

It is possible, for example, for the coil holder to support the coil device and include a spacer or for a spacer that is designed to be separate from the coil holder to be used, wherein the free volume is maintained by restricting the free volume at least in sections using the spacer, wherein the coil device and/or the coil holder are resting on the side of the spacer that is turned away from the free volume. A spacer that is designed to be separate from the coil holder can be configured as a supporting unit or supported by the mounting or at least by a further mounting. The spacer can be arc-shaped or u-shaped and in particular elastically dimensionally stable. It is also possible for the spacer to be deformed by the supported coil holder or the coil device, and to exercise a restoring force on the coil holder and/or the coil device, which prevents the coil holder and/or the coil device from penetrating the free volume.

Prior to being arranged on the examination object, the coil device and/or the coil holder can be encased in a sterilizable cover. As a result thereof, it is possible to use a coil holder or a coil device that are not themselves suitable for use in a sterile area, even if sterility is required. It is possible here to use a sterilizable cover that is elastically dimensionally stable. In this case the coil holder and/or the coil device can be given elastic dimensional stability by means of a sterilizable cover, thus allowing, for example, a free volume to be maintained without additional spacers.

Two free ends of the coil holder or of the coil device or one free end of a coil holder or a coil device and a free end of a further coil holder or of a further coil device can be maintained in a predetermined position by having connecting sections of the sterilizable cover or covers that encase the respective ends fixed together prior to the magnetic resonance-based examination step. The connecting sections can for example, be designed to be self-adhesive. Such a connection using connecting sections is particularly advantageous if, as explained in the aforementioned, free sections of the coil holder and/or of the coil device are meant to be bent and affixed in order to encompass the examination object.

In addition to the method, one or more of the present embodiments relate to a supporting device for at least one coil holder, which is designed to be used in the method described above, wherein the supporting device can be arranged between the object table and the transfer plate. As a mounting, the supporting device can comprise preferably at least one recess into which the coil holder engages. The supporting device preferably comprises a mounting on two opposite side surfaces. The mountings can for example, each be rectangular recesses. The supporting units can consist, for example, of foam or plastic.

The supporting unit can include an elastically flexible spacer which is equipped to counteract a bending of a coil holder that is supported in the supporting unit with a restoring force. For example, the spacer can be configured as an elastically bendable strip which, in an arrangement of the supporting unit between the object table and the transfer plate, extends above the mounting, such that a coil holder which is supported by the mounting, if it is bent and fastened in order to encompass an examination object, is pushed away from a free volume by a restoring force in the spacer.

One or more of the present embodiments additionally relate to a transfer plate to position an examination object, which plate is configured to be used in the aforementioned method, wherein the transfer plate has the mounting on at least one of the lateral surfaces that are different from the upper surface. In particular, the transfer plate can include a patient mattress and/or a head rest, which form the upper side or a part of the upper side of the transfer plate, the mounting being arranged on a side surface that is different from the upper surface of the patient mattress or the head rest. Preferably, the transfer plate, the patient mattress or the head rest have two mountings on opposite side surfaces.

One or more of the present embodiments further relate to a coil holder, which is configured to be used in the aforementioned method. The coil holder can be arc-shaped. It can additionally or alternatively be equipped to support at least one coil device, wherein it encompasses at least one mounting groove or a u-shaped mounting section to accommodate a free end of the coil device or of a fixing element to connect the coil device with the coil holder. A fixing peg of a fixing strip, for example, can be accommodated as a fixing element, with which the coil device can be affixed onto the coil holder. In the case of an arc-shaped coil holder, the u-shaped mounting section is preferably arranged on the outside of the arc. For example, two u-shaped mounting sections can be arranged on the coil holder in order to support two free ends of a coil device.

Rotatable fixing hooks can be arranged on the coil holder, which hooks are equipped to engage in recesses of the coil device if said device is supported by the coil holder.

The coil holder can be configured in a plurality of parts, wherein a component supporting the local coil or the coil unit can be moved with respect to the supporting section of the coil holder in at least one direction. A movement thereof can be in a longitudinal and/or transverse direction parallel to the upper side of the transfer plate and/or vertical to the upper side of the transfer plate.

As already explained with respect to the method according to one or more of the present embodiments, the coil holder can include a spacer.

The coil holder can include two supporting sections that are connected by an arc-shaped connecting section. The connecting section can be dimensionally stable, in particular elastically dimensionally stable and/or act as a spacer for a coil device supported by the coil holder.

One or more of the present embodiments additionally relate to a spacer, which is designed to be used in the method as the spacer that is designed to be separate from the coil holder. The spacer can essentially be arc-shaped and extend over at least one section of the transfer plate. The spacer can comprise two supporting sections which are designed to be connected with mountings on the transfer plate, the supporting unit, and/or the object table.

The spacer can comprise at least one u-shaped mounting section to accommodate a free end of a coil device or of a coil holder. This is particularly advantageous if, as explained with respect to the method, the coil holder and/or the coil device is/are bent or fastened such that it encompasses or they encompass the examination object at least partially. In the case of a u-shaped spacer, the u-shaped supporting section is preferably arranged on the outer side of the arc. The spacer can comprise two u-shaped supporting sections, the apertures of which are arranged in different directions and which preferably complete each other to form an s-shaped projection to accommodate two different ends of the same coil holder or of the same coil carrier or ends of two coil holders or coil devices.

One or more of the present embodiments also relate to a magnetic resonance unit, which includes a supporting unit and/or a transfer plate and/or a coil holder and/or a spacer.

### BRIEF DESCRIPTION OF THE DRAWINGS

FIGS. 1 to 5 show various possibilities of the mounting of the coil holder in various exemplary embodiments of a method;

FIGS. 6 to 8 show various possibilities of the mounting of coil devices on coil holders in various exemplary embodiments of the method;

FIG. 9 shows an exemplary embodiment of a spacer; and

FIG. 10 shows an exemplary embodiment of a coil holder.

### DETAILED DESCRIPTION

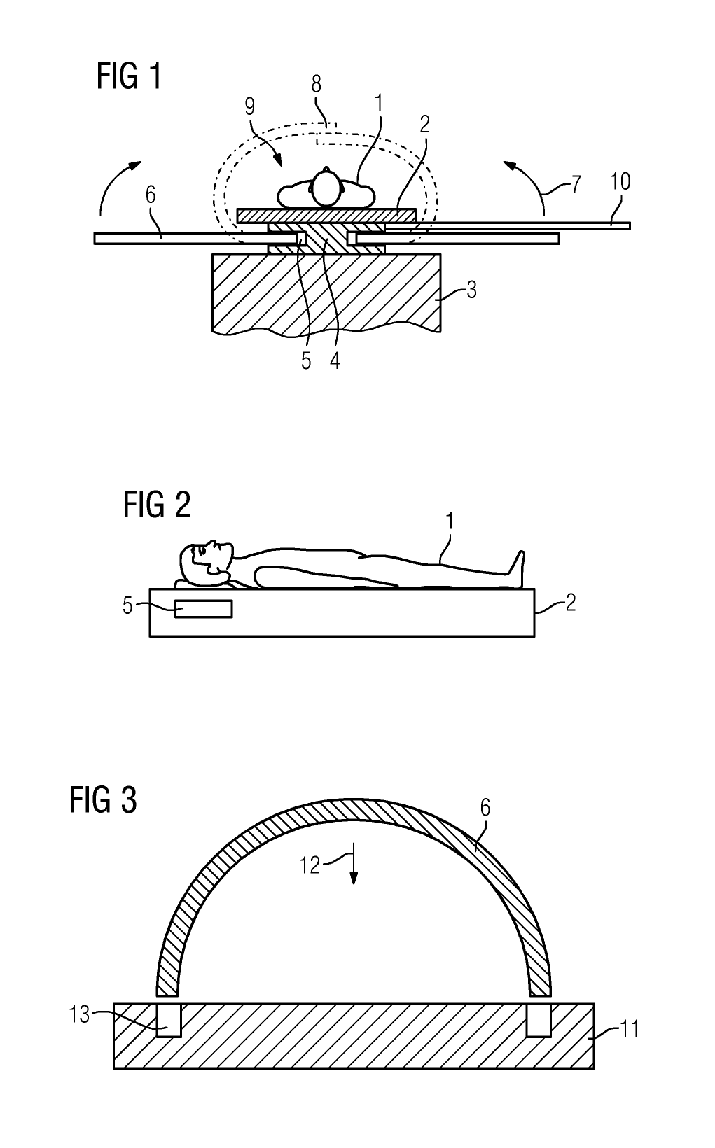

FIG. 1 shows a possibility for arranging local coils on an examination object 1, when various examinations are being performed on the object and local coils are to be arranged on the examination object 1 only during a magnetic resonance-based examination. The examination object 1 is positioned on the transfer plate 2 during the entire examination. This is shown as a simple plate, but the transfer plate 2 may include a patient mattress and/or a head rest may be provided in the area of the head of the examination object 1. The plate is narrower in the transverse direction in FIG. 1 than the transfer plate 2 in the other areas. These elements are not shown for reasons of clarity.

Prior to the magnetic resonance-based examination, the transfer plate 2 is intended to be arranged on the object table 3. In order to allow a simple arrangement of the local coils, prior to this step, however, the supporting unit 4 is arranged on the object table 3. The supporting unit 4 may be configured to be separate from the transfer plate 2. In this way, in a later step, the transfer plate may be moved over the supporting unit 4. The supporting unit 4 includes two mountings 5 arranged on opposite side surfaces of the supporting unit 4. Inserted into each mounting 5 is a supporting section of a coil holder 6 that includes a plurality of local coils. The coil holder 6 may be, for example, a Flex coil, in which a plurality of coils are arranged in an elastically dimensionally stable housing.

After arranging the supporting unit 4 together with the coil holders 6 fitted thereto, the examination object 1 is positioned on the transfer plate 2 on the object table 3. The supporting unit 4 is therefore arranged between the object table 3 and the transfer plate 2.

The free sections of the coil holder 6 are subsequently bent in the direction of the arrow 7 into the position shown with a dotted line 8 and fastened there, for example, by sticking together self-adhesive areas provided on the coil holders or by hooking the coil holders to each other or by using a further element.

When the right-hand coil holder 6 is bent, an elastically bendable spacer 10 arranged on the supporting unit 4 is automatically bent too. The spacer exerts a righting moment to bring the coil holder 6 back into its original position. If the coil holder 6 is bent together with the coil holder 6 until the coil holder 6 assumes the position 8, then the spacer 10 exerts a force on the coil holder 6 that pushes the holder 6 out of a free volume 9. Consequently, the free volume 9 is maintained in order for example, to allow intubation of the examination object 1.

FIG. 2 shows an alternative option for mounting the coil holder 6. Instead of the supporting unit 4, a transfer plate 2 that has a recess in the head area of the examination object 1 is used. The recess acts as a mounting 5 for the coil holders 6. The coil holders 6 may, for example, as explained in relation to FIG. 1, be inserted into this mounting 5 in order to then be bent round the examination object 1 and fixed in a final position. Using the transfer plate 2 shown in FIG. 2 makes it possible to arrange the local coils on the examination object 1 even prior to the object being transferred to the object table 3.

FIG. 3 shows a further option for mounting the coil holder 6. In this exemplary embodiment, the coil holder 6 is arc-shaped and is intended to be supported on a component of the transfer plate 11 (e.g., on an extension of the head area). For this purpose, the transfer plate 11 has two mountings 13 that are configured as grooves, for example. Alternatively, the mountings may also be configured as simple slits or drill holes. Prior to the magnetic resonance-based examination step, the coil holder 6 is arranged on the examination object, not shown for reasons of clarity, by the coil holder 6 being guided in the direction of the arrow 12 onto the examination object positioned on the transfer plate 11. The supporting sections of the coil holder 6 are inserted into the mountings 13.

The exemplary embodiment according to FIG. 3 may deviate from this in that the mountings 13 in the form of grooves are provided not as a part of the transfer plate 11 but on an object table or on a supporting unit that is arranged between the object table and the transfer plate and protrudes over the transfer plate. The placement of the coil holder, as described, allows a particularly simple attachment of local coils.

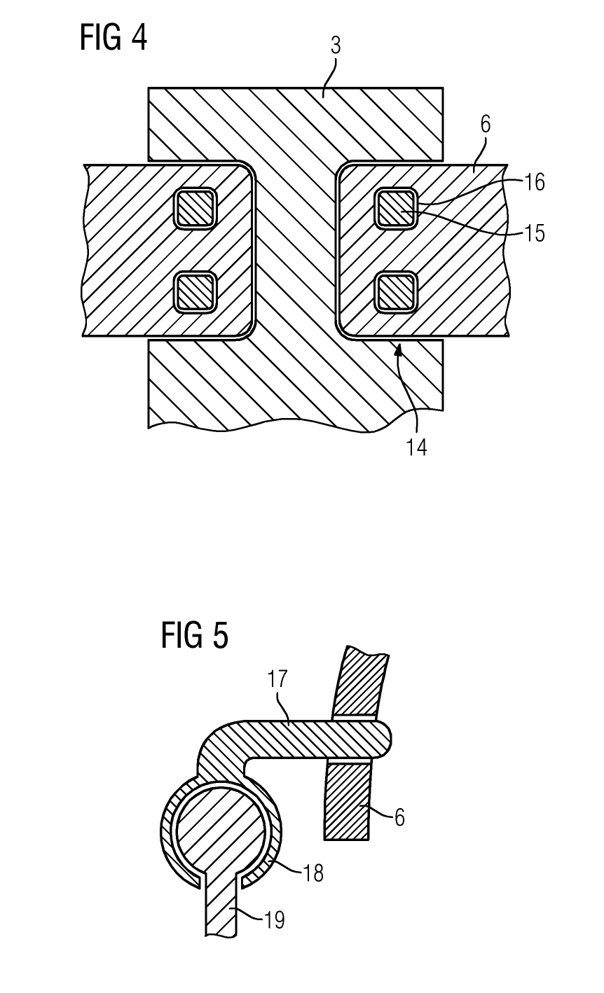

FIG. 4 shows a further option for the arrangement of the coil holders on the examination object. Mountings 15 arranged on the upper side of the object table 3 (e.g., engage into a supporting section of the respective coil holders 6) are used. Prior to the transfer plate being transferred onto the object table 3, the coil holders 6 are placed in recesses 14 in the object table 3 and are supported there by the mountings 15, which engage in recesses 16 perpendicular to the image plane of the coil holders 6. After positioning the transfer plate on the object table 3, the coil holders 6 may, for example, be bent round the examination object 1, as explained in FIG. 1.

In an alternative embodiment, the supporting units may be used on the object table 3 in order to support a coil holder that essentially rests flat on the object table 3 underneath the transfer plate. In this case, additional local coils may, for example, be arranged directly on the examination object 1 above the transfer plate, and/or further coil holders may be provided to support the coils.

FIG. 5 shows a further option for supporting a coil holder 6. In this exemplary embodiment, the coil holder 6 includes a supporting section 17 that at a free end has an elastically dimensionally stable pincer element 18. In this case, the mounting 19 is configured as a protrusion that becomes wider at a free end. Through this arrangement, the mounting 19, together with the supporting section 17, may fit into a releasable locking connection.

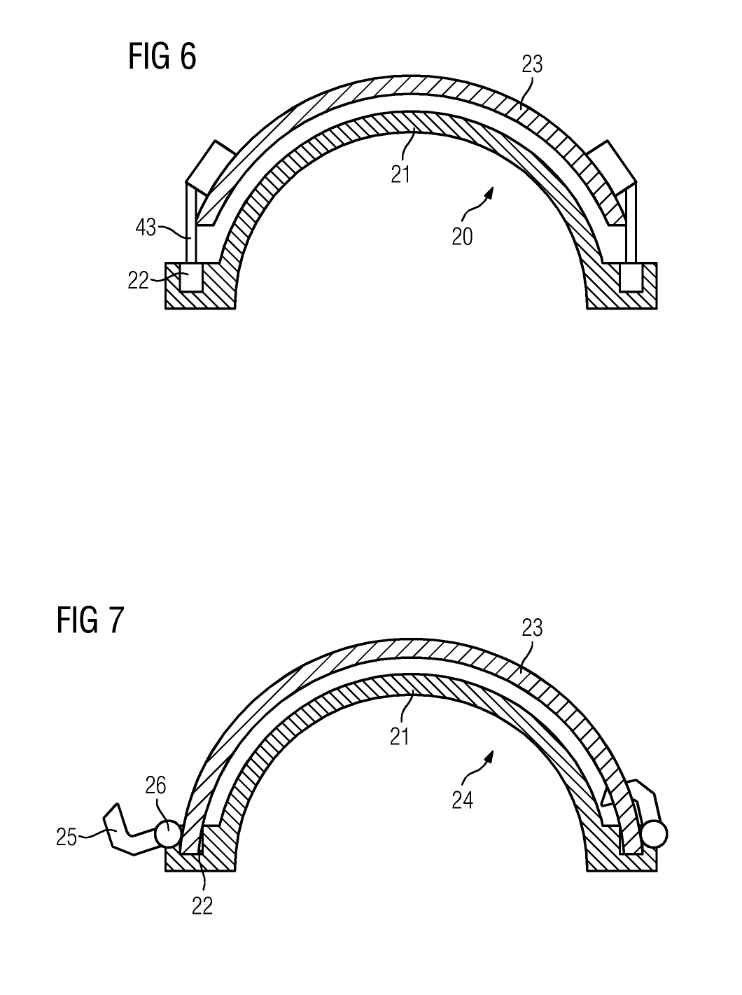

In the simplest scenario, the coil holder includes local coils. In alternative embodiments, a coil holder, by which at least one coil device that includes at least one local coil is supported, is used. An example thereof is shown in FIG. 6. The coil holder 20 includes two fixing grooves 22 that accommodate a fixing element (e.g., a fixing pin of a fixing strip 43 located on the coil device). Through the fixing strip 43, the coil device 23 is connected to the coil holder 20 on both sides. The coil device 23 itself is elastic and is bent over the fixing strips 43 in an arc. In order to preserve the arc-shaped form of the coil device 23 and therefore create a free volume for the examination object and, for example, for intubation of the examination object, the coil holder 20 also includes a spacer 21 configured as a relatively stiff arc that connects the two fixing grooves 22. The arc-shaped form of the spacer 21 provides that the coil device 23 does not penetrate a free volume above the transfer plate (which is not shown). The coil holder 20 may be fixed by fixing sections that are not shown to mountings on the object table, the transfer plate, or the supporting unit, as explained in the aforementioned.

The coil holder 24 shown in FIG. 7 is mainly configured like the coil holder 20 shown in FIG. 6. The fastening of the coil device 23 onto the coil holder 24 ensues in a slightly different way, however. The fixing grooves 22 in the coil holder 24 each accommodate a free end of the coil device 23. In order to provide a stable mounting of the coil device 23 even when the coil device is bent considerably, rotatable fixing hooks 25 are arranged on the sides of the coil holder 24. The rotatably fixing hooks 25 are rotatable round an axis 26 that is perpendicular to the image plane in order to engage in recesses, which are not shown, of the coil device 23.

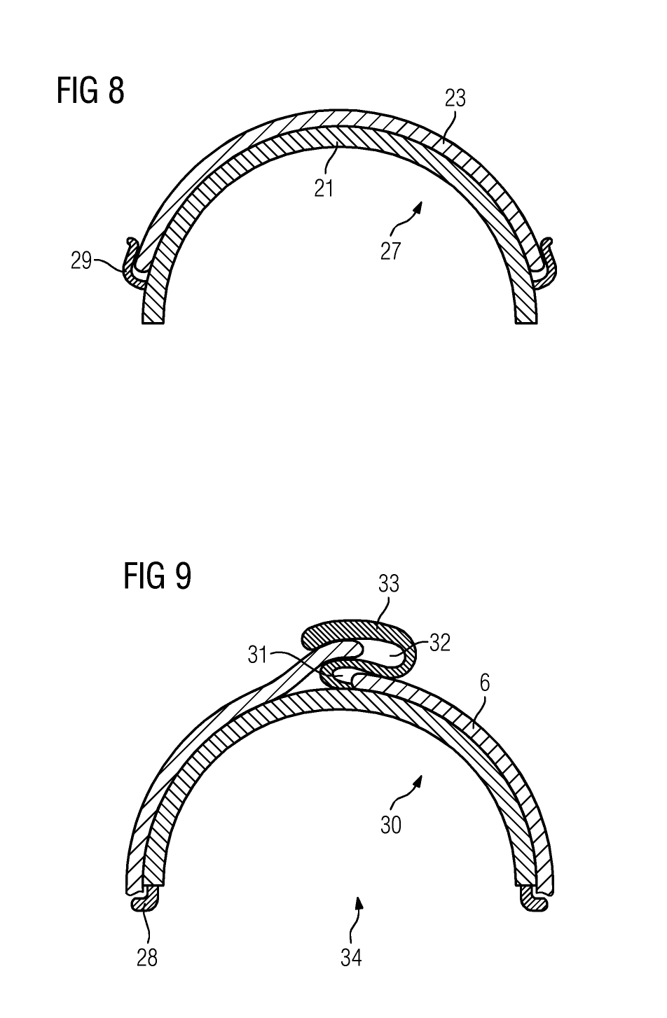

A further option for fastening a coil device to a coil holder is shown in FIG. 8. On the outer side of the arc-shaped spacer 21, the coil holder 27 has two u-shaped mounting sections 29, with which the coil device 23 may be mounted in each case.

FIG. 9 shows a spacer that is configured to be separate from a coil holder. The coil holders 6 are supported outside the area image area by mountings. A corresponding mounting may be achieved, for example, as shown in FIG. 1, 2, 4 or 5. For example, in cases in which the coil holders 6 have only slight dimensional stability in a final position, a spacer 30 may also be used. The spacer 30 may be mounted on supporting sections 28 on further mountings that may be arranged on the object table, the supporting unit, or the transfer plate. The spacer is arc-shaped in design and delimits a free volume 34 beside the examination object. The coil holders 6 sit closely on the side of the spacer 30 that is turned away from the free volume 34. In order to support the coil holders 6, two u-shaped mounting sections 31, 32 are arranged on the outside of the arc. Together these form an s-shaped projection 33 and allow the coil holders 6 to overlap for improved imaging.

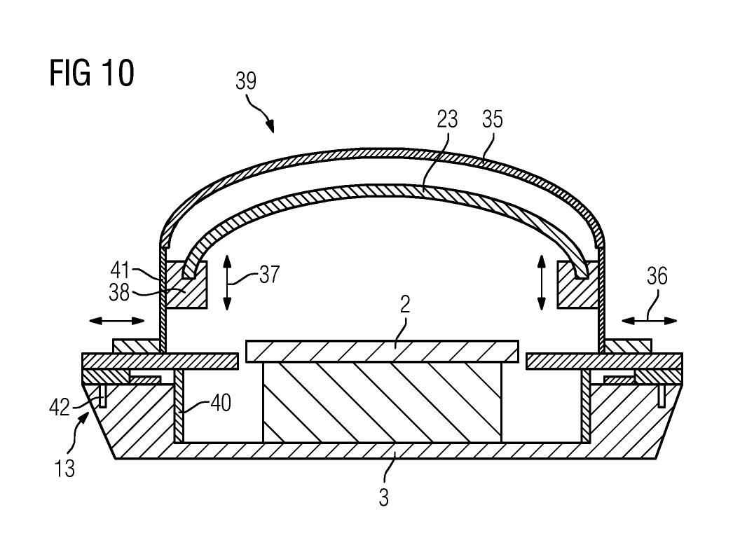

FIG. 10 shows an exemplary embodiment of a coil holder 39, which is fastened on the object table 3 by supporting sections 42 of the coil holder 39 that engage in mountings 13 of the object table 3. For additional stabilization, the coil holder 39 is supported by the protrusions 40 at further points on the object table 3, but this additional support may be dispensed with, however. The coil holder 39 is formed of a plurality of parts. A first component is fixedly connected to the respective supporting sections 42. A further component 41 is placed thereon such that the further component 41 is moveable in the direction of the arrow 36. In the exemplary embodiment shown, the components 41 that are moveable in this way are connected by an additional arc-shaped connecting section 35. This may be dispensed with in other exemplary embodiments, however. On the component 41 is placed a further component 38 that may be moved in the direction of the arrow 37 with respect to the component 41 and on which the coil device 23 is supported. Using the configuration shown, sliding of the coil device 23 with respect to the supporting sections 42 is possible in FIG. 10 in both an upward and a transversal direction. A sliding thereof perpendicular to the imaging plane is facilitated by the fact that the mounting 13 is designed as a groove, into which a straight supporting section 42 of the coil holder 39 engages. Consequently, the coil holder 39 may be moved inside the groove perpendicular to the image direction. The position of the coil device 23 in the upward and/or transversal direction and/or perpendicular to the imaging plane may be fixed, for example, by a locking connection, as shown in FIG. 5.

Although the invention has been illustrated and described in greater detail with the exemplary embodiments, the invention is not restricted to the examples disclosed. Other variants may be derived therefrom by a person skilled in the art without going beyond the scope of the invention.

The elements and features recited in the appended claims may be combined in different ways to produce new claims that likewise fall within the scope of the present invention. Thus, whereas the dependent claims appended below depend from only a single independent or dependent claim, it is to be understood that these dependent claims may, alternatively, be made to depend in the alternative from any preceding or following claim, whether independent or dependent. Such new combinations are to be understood as forming a part of the present specification.

While the present invention has been described above by reference to various embodiments, it should be understood that many changes and modifications can be made to the described embodiments. It is therefore intended that the foregoing description be regarded as illustrative rather than limiting, and that it be understood that all equivalents and/or combinations of embodiments are intended to be included in this description.

1. A method for performing an examination that includes at least one magnetic resonance-based and at least one further examination step, the method comprising:
positioning an examination object during an entire examination on an upper side of a transfer plate that is positioned on an object table during the magnetic resonance-based examination,
wherein at least one coil holder that includes at least one local coil or by which at least one coil device is supported is used during the magnetic resonance-based examination step,
wherein the at least one coil holder is arranged on the examination object exclusively during the magnetic resonance-based examination step,
wherein the at least one coil holder is supported during the magnetic resonance-based examination step by at least one mounting into which a supporting section of the at least one coil holder engages, that engages into the supporting section of the at least one coil holder, or a combination thereof, and
wherein the mounting is configured as part of the object table, the transfer plate, or a supporting unit that is arranged between the object table and the transfer plate.

2. The method of claim 1, wherein a mounting of the at least one mounting is configured on a side surface of the transfer plate that differs from the upper side.

3. The method of claim 1, wherein prior to the magnetic resonance-based examination step, the method further comprises arranging the at least one coil holder on the examination object, the arranging comprising guiding vertically or at an angle to the upper side of the transfer plate in a direction of the examination object, and
wherein the supporting section is inserted into the mounting, or the mounting is inserted into the supporting section.

4. The method of claim 1, wherein the mounting and the supporting section are connected by a releasable locking connection.

5. The method of claim 1, wherein prior to the magnetic resonance-based examination step, the method further comprises moving the at least one coil holder in the mounting or the mounting together with the at least one coil holder supported thereon with respect to the examination object in parallel with the transfer plate.

6. The method of claim 1, wherein prior to the magnetic resonance-based examination step, the method further comprises bending and fixing at least one free section of the at least one coil holder, the at least one coil device, or the at least one coil holder and the at least one coil device such that the at least one coil holder, the at least one coil device, or the at least one coil holder and the at least one coil device alone, together, or alone and together with the object table, the transfer plate, or the object table and the transfer plate surround the examination object during the magnetic resonance-based examination step.

7. The method of claim 1, wherein the at least one coil holder, the at least one coil device, or the at least one coil holder and the at least one coil device are arranged on the examination object such that the at least one coil holder, the at least one coil device, or the at least one coil holder and the at least one coil device alone, together, or alone and together with the object table, the transfer plate, or the object table and the transfer plate encompass the examination object during the magnetic resonance-based examination step, and
wherein on a side of the examination object that is turned away from the transfer plate, a predetermined free volume is maintained between the examination object and the at least one coil holder, the at least one coil device, or the at least one coil holder and the at least one coil device.

8. The method of claim 7, wherein the at least one coil holder supports the at least one coil device and encompasses a spacer, or a spacer that is configured separately from the at least one coil holder is used,
wherein the predetermined free volume is maintained by delimiting the free volume at least in sections by the spacer,
wherein the at least one coil device, the at least one coil holder, or the at least one coil device and the at least one coil holder rest at least in sections on a side of the spacer that is turned away from the free volume.

9. The method of claim 7, wherein prior to the arrangement of the at least one coil device, the at least one coil holder, or the at least one coil device and the at least one coil holder on the examination object, the at least one coil device, the at least one coil holder, or the at least one coil device and the at least one coil holder are encased in a sterilizable cover.

10. The method of claim 9, wherein the sterilizable is elastically dimensionally stable.

11. The method of claim 9, wherein two free ends of the at least one coil holder, the at least one coil device, or the at least one coil holder and the at least one coil device, or one free end of a coil holder of the at least one coil holder or of a coil device of the at least one coil device and a free end of a further coil holder of the at least one coil holder or of a further coil device of the at least one coil device are maintained in a predetermined position by having connecting sections of the respective sterilizable cover that encases the respective ends fixed together prior to the magnetic resonance-based examination step.

12. A mounting device for at least one coil holder, the mounting device being usable in performance of an examination that includes at least one magnetic resonance-based and at least one further examination step, the performance of the examination comprising an examination object being positioned during an entire examination on an upper side of a transfer plate that is positioned on an object table during the magnetic resonance-based examination, wherein at least one coil holder that includes at least one local coil or by which at least one coil device is supported is used during the magnetic resonance-based examination step, wherein the at least one coil holder is arranged on the examination object exclusively during the magnetic resonance-based examination step, the mounting device comprising:
at least one mounting configured to support the at least one coil holder during the magnetic resonance-based examination step, engage with a supporting section of the at least one coil holder, or a combination thereof,
wherein the at least one mounting is configured as part of the object table, the transfer plate, or a supporting unit that is arranged between the object table and the transfer plate.

13. The mounting device of claim 12, wherein the at least one mounting comprises mountings on two opposite side surfaces.

14. The mounting device of claim 12, wherein the supporting unit includes an elastically bendable spacer configured to counteract any bending of the at least one coil holder that is supported in the at least one mounting with a restoring force.

15. A transfer plate for positioning an examination object, the transfer plate being usable in an examination that includes at least one magnetic resonance-based and at least one further examination step, the transfer plate comprising:
an upper side on which an examination object is positionable during an entire examination, the transfer plate being positionable on an object table during the magnetic resonance-based examination, wherein at least one coil holder that includes at least one local coil or by which at least one coil device is supported is usable during the magnetic resonance-based examination step, wherein the at least one coil holder is arrangeable on the examination object exclusively during the magnetic resonance-based examination step; and
a mounting on at least one side surface that is different from the upper surface,
wherein the at least one coil holder is supportable during the magnetic resonance-based examination step by the mounting, into which a supporting section of the at least one coil holder engages, that engages into the supporting section of the at least one coil holder, or a combination thereof.

16. A coil holder usable in an examination that includes at least one magnetic resonance-based and at least one further examination step, the examination including an examination object being positioned during an entire examination on an upper side of a transfer plate that is positioned on an object table during the magnetic resonance-based examination, the coil holder comprising:
at least one local coil that is usable during the magnetic resonance-based examination step,
wherein the coil holder is arrangeable on the examination object exclusively during the magnetic resonance-based examination step,
wherein the coil holder is supportable during the magnetic resonance-based examination step by at least one mounting, into which a supporting section of the coil holder engages, that engages into the supporting section of the coil holder, or a combination thereof, and
wherein the mounting is configured as part of the object table, the transfer plate, or a supporting unit that is arranged between the object table and the transfer plate.

17. The coil holder of claim 16, wherein the coil holder is configured to support at least one coil device,
wherein the coil holder has at least one fixing groove or a u-shaped mounting section to accommodate a free end of the at least one coil device or of a fixing element to connect the at least one coil device to the coil holder.

18. The coil holder of claim 17, further comprising at least one rotatable fixing hook arranged on the coil holder,
wherein the at least one rotatable fixing hook is configured to engage in recesses of the at least one coil device when the at least one coil device is supported by the coil holder.

19. The coil holder of claim 17, wherein the coil holder is configured to have a plurality of parts, and
wherein a component supporting the local coil or the at least one coil device is movable with respect to the supporting section of the coil holder in at least one direction.

20. The coil holder of claim 17, further comprising two supporting sections that are connected by an arc-shaped connecting section.

21. A spacer usable in an examination that includes at least one magnetic resonance-based and at least one further examination step, the examination comprising an examination object being positioned during an entire examination on an upper side of a transfer plate that is positioned on an object table during the magnetic resonance-based examination, wherein at least one coil holder that includes at least one local coil or by which at least one coil device is supported is usable during the magnetic resonance-based examination step, wherein the at least one coil holder is arrangeable on the examination object exclusively during the magnetic resonance-based examination step, wherein the at least one coil holder is supportable during the magnetic resonance-based examination step by at least one mounting into which a supporting section of the at least one coil holder engages, that engages into the supporting section of the at least one coil holder, or a combination thereof, and wherein the mounting is configured as part of the object table, the transfer plate, or a supporting unit that is arranged between the object table and the transfer plate, wherein the spacer is configured to be separate from the at least one coil holder,
wherein the spacer is configured to delimit a free volume at least in sections, such that the predetermined free volume is maintained, and
wherein the at least one coil device, the at least one coil holder, or the at least one coil device and the at least one coil holder rest at least in sections on a side of the spacer that is turned away from the free volume.

22. The spacer of claim 21, further comprising at least one u-shaped mounting section to accommodate a free end of a coil device or of a coil holder.
